(19) United States
(12) Patent Application Publication
(10) Pub. No.: US 2004/0142914 A1
(43) Pub. Date: Jul. 22, 2004

(54) Extended transdermal contraceptive regimens

(76) Inventors: Andrew Joseph Friedman, Princeton, NJ (US); Katherine D. LaGuardia, New York, NY (US)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/626,434

(22) Filed: Jul. 24, 2003

Related U.S. Application Data
(63) Continuation-in-part of application No. 10/385,597, filed on Mar. 11, 2003.
(60) Provisional application No. 60/363,167, filed on Mar. 11, 2002.
(60) Provisional application No. 60/381,585, filed on May 17, 2002.

(51) Int. Cl. A61K 31/56

(52) U.S. Cl. 514/170; 424/449

(57) ABSTRACT

A method of contraception is described comprising the step of administering to a menstruating female a cycle of contraceptive therapy, said cycle of therapy including, for at least 42 successive days, the administration of a combination of an estrogen and a progestogen in a contraceptively effective daily dosage wherein said progestogen is a potent sulfatase inhibiting progestogen and said cycle of therapy including 4-8 days which are free of estrogen administration following said at least 42 successive days. A method of contraception is also described which provides enhanced bleeding control and enhanced continuation and satisfaction rates in menstruating females using the method.

### RELATED APPLICATION

This application is a continuation-in-part of prior application Ser. No. 10/385597, filed on Mar. 11, 2003, which claims the benefit under 35 U.S.C. § 119(e) of provisional applications Serial Nos. 60/363,167 and 60/381,585, filed on Mar. 11, 2002 and May 17, 2002, respectively.

The present invention relates to extended cycle contraceptive regimens for menstruating females. More particularly, the present invention relates to extended cycle contraceptive regimens containing a potent sulfatase inhibiting progestogen, such as, norgestimate (NGM) or norelgestromin (NGMN), and an estrogen.

### BACKGROUND OF THE INVENTION

A substantial percentage of human breast carcinomas are hormone-dependent. Animal studies and clinical trials have confirmed that estrogens, particularly estradiol, are the most important hormones involved in supporting growth of hormone-dependent breast tumours. (see refs #1 at 493, #2 at 967, #7 at 1589, #8 at 525, #9 at 135, #10 at 225, #11 at 625 and #12 at 1497)

Plasma levels of estrone and estradiol in post-menopausal women are very low. (see refs #1 at 493 and #11 at 626) Yet, breast tumor tissue concentration of estrone and estradiol is an order of magnitude higher than plasma concentrations. (see refs #1 at 493, #2 at 967 and #13 at 641) FIG. 1 shows the enzymatic process by which estrogens are locally formed in human breast cancer cells and thereby made available to support growth. (see ref #10 at 229). Referring to FIG. 1, studies have shown that the sulfatase enzyme appears to be at least 10× more important in the formation of estrogens than the aromatase enzyme. (see refs #1 at 493, #2 at 967, #4 at 17, #5 at 931, #7at 1589, #8at 525, #9at 135, #10at 228, #11 at 626 and 628 and #13 at 641) Thus, it is the sulfatase pathway that is the primary pathway promoting local formation of estrogens in human breast cancer cells.

Since estradiol is one of the main factors involved in supporting growth of hormone-dependent breast tumours and the sulfatase pathway is the main pathway for the formation of estradiol in the breast, then a decrease of estradiol formation by suppression of the sulfatase pathway would have potential therapeutic activity in the management of breast cancer. (see refs #1 at 493, #3 at 55, #4 at 17, #5 at 931, #6 at 123 and #11 at 631) Suppression of the sulfatase pathway will have a breast protective effect.

Local formation of estrogens in the breast is only one source for exposure of breast tissues to estrogens. Another source of estrogen present in breast tissues is estrogen containing contraceptive regimens. Most such regimens follow a cycle of 28 days including 7 days without administration of a hormone, including estrogen, preceded by 21 days of combined administration of progestogen and estrogen. There is presently an increased interest in regimens of longer than 28 days. Such regimens would have extended cycles of 6 to 26 weeks, such as 6, 8, 12 or 13 weeks. In such extended cycles, the period of hormone free or estrogen free administration would not increase over the hormone free or estrogen free period of 28 day cycle regimens. Thus, a 91 day cycle would include 7 days without administration of a hormone, including estrogen, preceded by 84 days of combined administration of progestogen and estrogen. As compared to a 28 day cycle, a 91 day cycle would require the administration of an estrogen for 84 of 91 days rather than 21 of 28 days. On a yearly basis this would mean 4 weeks without estrogen administration for the 91 day cycle as compared to 13 weeks without estrogen administration for the 28 day cycle. The increased exposure to estrogen is recognized as a possible disadvantage (see refs #14 at 275 and #15 at 94).

It is an object of the present invention to provide an extended cycle contraceptive regimen to continuously suppress sulfatase activity in human breast cancer cells.

It is also an object of the present invention to provide an extended cycle contraceptive regimen with exceptional suppression of sulfatase activity in human breast cancer cells.

It is also an object of the present invention to provide an extended cycle contraceptive regimen to continuously suppress estrogen formation in human breast cancer cells.

It is yet another object of the present invention to provide an extended cycle contraceptive regimen with exceptional suppression of estrogen formation in human breast cancer cells.

It is still another object of the present invention to provide an extended cycle contraceptive regimen which minimizes exposure of the breast to locally formed estrogen.

It is another object of the present invention to provide an extended cycle contraceptive regimen which reduces exposure of the breast to estrogens as compared to other extended cycle contraceptive regimens of equivalent estrogen dose.

It is another object of the present invention to provide an extended cycle contraceptive regimen with the lowest levels of breast estrogen exposure as compared to other extended cycle contraceptive regimens of equivalent estrogen dose.

It is another object of the present invention to provide an extended cycle contraceptive regimen which closely limits exposure of the breast to those levels of estrogens which are administered in the regimen or produced in vivo outside the breast.

It is still another object of the present invention to provide an extended cycle contraceptive regimen which provides exceptional and continuous breast protective effect.

It is another object of the present invention to provide an extended cycle contraceptive regimen which minimizes risk factors associated with breast cancer.

References:

1. Inhibition of Estrone Sulfatase Enzyme in Human Placenta and Human Breast Carcinoma; T. R. JEFFRY EVANS et al., J. Steroid Biochem. Molec. Biol. Vol. 39, No. 4A 1991, pp. 493-499.

2. In Vitro Effect of Synthetic Progestogens on Estrone Sulfatase Activity in Human Breast Carcinoma; ODILE PROST-AVALLET et al., J. Steroid Biochem. Molec. Biol., Vol. 39, No. 6, 1991, pp. 967-973.

3. Effect of the progestagen R5020 (promegestone) and of progesterone on the uptake and on the transformation of estrone sulfate in MCF-7 and T-47d human mammary cancer cells: correlation with progesterone receptor levels; JORGE R. PASQUALINI et al., Cancer Letters, 66 (1992) 55-60, Elsevier Scientific Publishers Ireland Ltd.

4. Action of Danazol On The Conversion Of Estrone Sulfate To Estradiol And On The Sulfatase Activity In The MCF-7, T-47D and MDA-MB-231 Human Mammary Cancer Cells; B-L NGUYEN et al., J. Steroid Biochem, Molec. Biol. Vol. 46, No. 1, 1993, pp. 17-23.

5. Effect of Promegestone, Tamoxifen, 4-Hydroxytamoxifen and ICI 164,384 on the Oestrone Sulphatase Activity of Human Breast Cancer Cells; GERARD CHETRITE et al., Anticancer Research 13: 931-934 (1993).

6. Inhibition Of Steroid Sulfatase Activity By Danazol; KJELL CARLSTROM et al., Acta Obstet Gynecol Scand Suppl. 107- 111.

7. Effect of the Progestagen Promegestone (R-5020) on mRNA of the Oestrone Sulphatase in the MCF-7 Human Mammary Cancer Cells; JORGE R. PASQUALINI et al., Anticancer Research 14: 1589-1594 (1994).

8. Effect of Nomegestrol Acetate on Estrone-sulfatase and 17β-Hydroxysteroid Dehydrogenase Activities in Human Breast Cancer Cells; G. CHETRITE et al., J. Steroid Biochem. Molec. Biol. Vol. 58, No. 5/6, pp. 525-531, 1996.

9. Effect of Tibolone (Org OD14) and its Metabolites on Estrone Sulphatase Activity in MCF-7 and T-47D Mammary Cancer Cells; G. CHETRITE et al., Anticancer Research 17: 135-140 (1997).

10. Progestins and Breast Cancer; J. R. PASQUALINI et al., J. Steroid Biochem. Molec. Biol. Vol. 65, No. 1-6, pp. 225-235, 1998.

11. Control of Estradiol In Human Breast Cancer. Effect of Medrogestone on Sulfatase, 17β-Hydroxysteroid Dehydrogenase And Sulfotransferase Activities in Human Breast Cancer Cells; JORGE RAUL PASQUALINI et al., Euro. Congr. On Menopause (1998), pp. 625-633.

12. Constitutive Expression of the Steroid Sulfatase Gene Supports the Growth of MCF-7 Human Breast Cancer Cells in Vitro and in Vitro; MATTIE R. JAMES et al., Endocrinology, Vol. 142, No. 4, pp 1497-1505.

13. Concentrations of Estrone, Estradiol and Their Sulfates, And Evaluation of Sulfatase and Aromatase Activities in Patients with Breast Fibroadenoma; J. R. PASQUALINI et al., Int. J. Cancer, 70, pp. 639-643 (1997).

14. A Trimonthly Regimen for Oral Contraceptives; G. T. KOVACS et al., The Brit. J. of Family Planning, 19, pp. 274-275 (1994)

15. Manipulation of Human Ovarian Function: Physiological Concepts and Clinical Consequences; B. C. J. M. FAUSER, Endocrine Review, Vol. 18, No. 1, (1997).

16. Continuous Combination Oral Contraceptive Pills to Eliminate Withdrawal Bleeding: A Randomized Trial; L. MILLER, et al., Obstetrics & Gynecology, Vol. 101, No. 4 (April 2003).

17. Bleeding Patterns and Patient Acceptability of Standard or Continuous Dosing Regimens of a Low-Dose Oral Contraceptive: A Randomized Trial; M. KWIECIEN, et al., Contraception, 67, pp. 9-13 (2003).

### SUMMARY OF THE INVENTION

According to the present invention there is provided, a method of contraception comprising the step of administering to a menstruating female a cycle of contraceptive therapy, said cycle of therapy including, for at least 42 successive days, the administration of a combination of an estrogen and a progestogen in a contraceptively effective daily dosage wherein said progestogen is a potent sulfatase inhibiting progestogen and said cycle of therapy including 4-8 days which are free of estrogen administration following said at least 42 successive days.

There is also provided by the present invention, a contraceptive therapy unit for administration to a menstruating female comprising a cycle of separate dosage units, said cycle of dosage units including at least 42 dosage units adapted for successive daily oral administration, wherein said dosage units contain, in admixture with a pharmaceutically acceptable carrier, a combination of an estrogen and a progestogen in a contraceptively effective daily dosage wherein said progestogen is a potent sulfatase inhibiting progestogen and, optionally, said cycle of dosage units including 4-8 dosage units containing no estrogen.

There is also provided by the present invention, a contraceptive therapy unit for administration to a menstruationg female comprising a cycle of transdermal patches, said cycle of transdermal patches including a sufficient number of patches adapted for successive administration to provide for at least 42 successive days of therapy, wherein said transdermal patches contain, in a suitable matrix, a combination of an estrogen and a progestogen for delivery in a contraceptively effective daily dosage wherein said progestogen is a potent sulfatase inhibiting progestogen and, optionally, said cycle of transdermal patches including a patch for 4-8 days of use containing no estrogen.

There is also provided by the present invention, a contraceptive therapy unit for administration to a menstruationg female comprising a cycle of vaginal rings, said cycle of vaginal rings including a sufficient number of rings adapted for successive administration to provide for at least 42 successive days of therapy, wherein the vaginal rings contain, in a suitable matrix, a combination of an estrogen and a progestogen for delivery in a contraceptively effective daily dosage wherein said progestogen is a potent sulfatase inhibiting progestogen and, optionally, said cycle of vaginal rings including a ring for 4-8 days of use containing no estrogen.

Applicants have surprisingly discovered that such a regimen is expected to have reduced levels of estrogen in the breast as compared to other extended cycle contraceptive regimens having equivalent doses of estrogens.

### BRIEF DESCRIPTION OF THE DRAWINGS

FIG. 1 shows the enzymatic process involved in the formation and transformation of estrogens in human breast cancers.

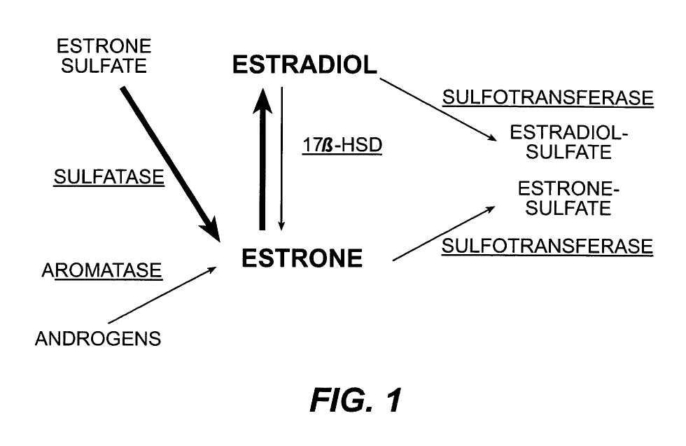

FIG. 2 illustrates a comparison of headache frequency for subjects who received cyclic transdermal administration of contraceptive hormones versus subjects who received extended transdermal administration of contraceptive hormones.

FIG. 3 illustrates a comparison of the percentage of subjects requiring sanitary protection for subjects who received extended oral administration of contraceptive hormones versus subjects who received extended transdermal administration of contraceptive hormones.

FIG. 4 illustrates a comparison of the mean number of days in each bleeding cycle that required sanitary protection for subjects who received extended oral administration of contraceptive hormones versus subjects who received extended transdermal administration of contraceptive hormones.

### DETAILED DESCRIPTION OF THE INVENTION

The contraceptive regimen according to the present invention is administered cycle after cycle to a menstruating female to achieve a long term contraceptive effect. Menstruating female is intended to refer to fertile women of child bearing age. The method of administration might be transdermal, vaginal or oral. Where administration is transdermal, a suitable patch is continuously worn with replacement as required. Where administration is vaginal, a suitable vaginal device, such as a ring, is continuously inserted with replacement as required. Where administration is oral, daily oral dosage units are administered.

Many common contraceptive regimens have a cycle of 28 days including 21 days of combined estrogen and progestogen administration followed by an off period of 7 days without administration of these hormones. Extended cycle contraceptive regimen, sometimes referred to herein as continuous regimen or administration, as these phrases are used herein, are intended to refer to contraceptive regimens having combined estrogen and progestogen administration of 42 days or longer followed by an off period of time without or with reduced administration of these hormones to allow menstruation. Thus, a minimum extended cycle would cover a period of time, which is 42 days of drug administration plus an off period of 4-8 days without drug or with reduced drug. A preferred extended cycle is 11 or 12 weeks of drug administration followed by an off period off 4-8 days without drug or with reduced drug. Another preferred extended cycle is 25 weeks of drug administration followed by an off period of 4-8 days without drug or with reduced drug.

The off period without or with reduced administration of hormone is to allow for menstruation as stated. During the off period there should be no estrogen administered. However, as can be understood from a general sense of the present invention, it may be desirable to continue the administration of a potent sulfatase inhibiting progestogen to obtain a continuation of its breast protecting effect. It would be desirable to continue progestogen administration to the extent that such administration does not interfere with menstruation. Therefore, it may be desirable to administer a full dose or reduced dose of progestogen for the full off period. A full dose is intended to mean a continuation of the dose administered in the active period of the cycle or of a progestogen dose named below as suitable for administration in the active period of the cycle. A reduced or minimized dose might be a tablet delivered oral norgestimate equivalent dose of 30 or 60 mcg or device delivered systemic circulation norgestimate equivalent dose of 18 or 30 mcg. Alternatively, it may be desirable to interrupt progestogen administration for a number of days less than the full off period. For example, there could be three days without estrogen or progestogen administration and for the remaining days of the off period there could be administered a full dose or reduced dose of progestogen. A preferred off period of time without or with reduced hormone to allow for menstruation is 7 days.

The extended cycle regimens herein may include a regimen in which there is a day to day or week to week variation in the dose of active administered according to a set pattern. In such a case the regimen, including variation of dose, is repeated in cycle following cycle. The extended cycle regimen may also be one in which there is no variation in the dose of the active administered. Whatever the case, an extended cycle contraceptive product utilizing the contraceptive regimen of the present invention is prescribed, sold and administered in units of cycles. The contraceptive product based on a cycle might be 4 to 25 vaginal rings that are inserted and then replaced every 7, 14 or 21 days according to their design. The contraceptive product based on a cycle might be 4 to 25 transdermal patches that are attached and then replaced every 7, 10 or 14 days according to their design. The contraceptive product based on a cycle might be 42, 49, 63, 84, 91, 126 or 182 tablets that are orally administered daily in a cycle that is 42/7, 49/7, 63/7, 84/7, 91/7, 126/7 or 182/7.

The estrogen in combination with the progestogen is administered in sufficient amounts to provide a contraceptive effect. Additionally, the estrogen dose in contraceptive regimens described herein is closely associated with the control of bleeding and spotting in the cycle. Between menstruations, bleeding and spotting should be minimized. Thus, 17α-ethinylestradiol might be also administered in sufficient amounts to control or minimize or eliminate bleeding and spotting during the inter-menstruation period of the cycle.

“Estrogen” herein refers to an estrogen receptor modulator having either an agonistic or antagonistic effect on the estrogen receptor, but preferably an agonistic effect. Any conventional estrogen may be employed as a suitable component in the contraceptive regimen of this invention. The particular estrogen employed should be selected and administered such that it is equivalent in contraceptive effect to a daily dosage of about 0.005-0.050 mg of 17α-ethinylestradiol. The preferred dosage of the estrogen employed is one equal to a daily dosage of about 0.010-0.035 mg of 17α-ethinylestradiol.

In addition to the commonly employed 17β-estradiol, there can be also be employed 17α-ethinylestradiol, esters and ethers of 17α-ethinylestradiol such as, for example, 17α-ethinylestradiol 3-dimethylamino propionate, 17α-ethinylestradiol 3-cyclopentyl ether (quienestrol) and 17α-ethinylestradiol 3-methyl ether (mestranol) as the estrogen component. Natural estrogens such as estrone, estrone sulfate, estrone sulfate piperazine salt, estradiol and estriol, and their esters, may also be employed. Conjugated equine estrogens (CEE) or conjugated estrogens (CE) are well known for this use. Suitable synthetic estrogens or synthetic estrogen modulators for use herein include tamoxifen, toremifene, ormeloxifene, modrefen, fulvestrant, lasofoxifene, bazedoxifene (TSE-424), arzoxifene, tesmilifene, miproxifene, EM-652 (Sch-57068), 3339 (Aventis), Ospemifene (Fc 1271A), ERA-923, GTx-006, HM-101, DPC-974, A-007, SP-8490, WAY-140424, tibolone, levodoxiphen, raloxifene.

In the case of a daily oral tablet, there is administered a preferred dose of 17α-ethinylestradiol (or contraceptively equivalent amount of a suitable estrogen) between about 0.005 mg to about 0.050 mg and more preferably between about 0.010 mg to about 0.035 mg. Specific daily oral tablets might contain 0.015, 0.020, 0.025 or 0.035 mg of 17α-ethinylestradiol. In the case of a vaginal ring, the preferred ring delivers to systemic circulation a daily dose of 17α-ethinylestradiol (or contraceptively equivalent amount of a suitable estrogen) between about 0.003 mg to about 0.030 mg and more preferrably between about 0.006 mg to about 0.020 mg. A specific vaginal ring might be inserted for one week and deliver to systemic circulation in that period of time an average daily dose of 0.009, 0.012, 0.015 or 0.020 mg of 17α-ethinylestradiol. In the case of a transdermal patch, a preferred patch delivers to systemic circulation a daily dose of 17α-ethinylestradiol (or contraceptively equivalent amount of a suitable estrogen) between about 0.003 mg to about 0.030 mg and more preferrably between about 0.006 mg to about 0.020 mg. A specific patch might be worn for one week and deliver to the surface of the skin in that period of time an average daily dose of 0.009, 0.012, 0.015 or 0.020 mg of 17α-ethinylestradiol. Regardless of the foregoing, it is intended herein to use conventional amounts of estrogen since it is not the estrogen component which is critical to the invention. Persons skilled in the art well understand required doses of estrogen required in contraceptive regimens.

A potent sulfatase inhibiting progestogen is preferably herein defined as a progestogen which has (or a progestogen with a substantial metabolite thereof which has) an IC₅₀ in the conversion of E₁S to E₂ in either the MCF-7 or T-47D breast cancer cell lines of about the corresponding IC₅₀ of norelgestromin or lower. A potent sulfatase inhibiting progestogen may also be a progestogen which has (or a progestogen with a substantial metabolite thereof which has) an IC₅₀ in the conversion of E₁S to E₂ in either the MCF-7 or T-47D breast cancer cell lines of substantially less than the corresponding IC₅₀ of medroxyprogesterone acetate, for example, on the order of ⅓, ½ or ⅕ of the IC₅₀ of medroxyprogesterone acetate. A potent sulfatase inhibiting progestogen can also be defined as a progestogen having (or a progestogen with a substantial metabolite thereof which has) an IC₅₀ in the conversion of E₁S to E₂ in either the MCF-7 or T-47D breast cancer cell lines of at most about 1/10, or about preferably 1/100, the corresponding IC₅₀ of medroxyprogesterone acetate (MPA). A potent sulfatase inhibiting progestogen can also be defined as a progestogen which inhibits (or a progestogen with a substantial metabolite thereof which inhibits) at least about 70% and preferably at least about 90% of the conversion of E₁S to E₂ in either the MCF-7 or T-47D breast cancer cell lines where employed in the test at a concentration of 50×10⁻⁶ mol/l.

Norgestimate (NGM) or norelgestromin (NGMN) are the preferred progestogens utilized herein and are each known to the art of contraceptive therapy. In fact, norgestimate is now used in a number of commercially available contraceptive products. The most preferred progestogen is norelgestromin (17-d-norgestimate). Norelgestromin is the major metabolite of norgestimate in humans with 80% and higher of norgestimate being converted to norelgestromin in vivo. For this reason, inhibition of sulfatase enzyme activity which is demonstrated for norelgestromin is inferred to norgestimate. Of course, to obtain equivalent inhibition of sulfatase enzyme activity (but not progestogenic effect), it may be necessary to administer a somewhat greater dose of norgestimate as compared to any dose of norelgestromin.

The progestogen is administered in conjunction with the estrogen in an amount sufficient to produce a contraceptive effect. The progestogen will also oppose the action of the estrogen on the endometrium. It has been observed that the long term administration of an estrogen which is unopposed by the administration of a progestogen leads to a substantial increase in the incidence of endometrial cancer. Thus, it is also desirable in a contraceptive regimen that the progestogen be administered in an amount which is an effective endometrium protective amount.

According to the present invention, it is now an additional requirement that the progestogen be administered in an amount which is an effective breast protective amount. More specifically, in a first characterization of a breast protective and otherwise suitable amount of progestogen, there is selected and administered sufficient sulfatase inhibiting progestogen such that it is at least equivalent in both contraceptive and breast protecting effect to about 0.030 mg to about 0.500 mg of orally administered norgestimate. Preferably, there is selected and administered sufficient sulfatase inhibiting progestogen such that it is at least equivalent in both contraceptive and breast protecting effect to about 0.050 mg to about 0.300 mg of orally administered norgestimate. In another characterization of a breast protective amount of progestogen and assuming a contraceptively effective amount, there is administered sufficient active compound to provide for, during a substantial portion of each day, a substantial supression of sulfatase activity, for example, of 50% or greater and preferably of 67% or greater and most preferably of 75% or greater. A substantial portion of a day is intended to mean a period of at least 4 hours, but within the invention might mean a period of at least 8 hours or 12 hours or even 24 hours. In the case of a daily oral tablet, there is administered a preferred dose of norgestimate or norelgestromin (or contraceptively equivalent amount of a suitable progestogen) between about 30 mcg to about 500 mcg and more preferably between about 50 mcg to about 300 mcg. Specific daily oral tablets might contain 125, 180, 215, 250 or 300 mcg of norgestimate or norelgestromin. In the case of a vaginal ring, a preferred ring delivers to systemic circulation a daily dose of norgestimate or norelgestromin (or contraceptively equivalent amount of a suitable progestogen) between about 18 mcg to about 300 mcg and more preferrably between about 30 mcg to about 175 mcg. A specific vaginal ring might be inserted for one week and deliver to systemic circulation in that period of time an average daily dose of 70, 100, 125, 140 or 175 mcg of norgestimate or norelgestromin. In the case of a transdermal patch, a preferred patch delivers to systemic circulation a daily dose of norgestimate or norelgestromin (or contraceptively equivalent amount of a suitable progestogen) between about 18 mcg to about 300 mcg and more preferrably between about 30 mcg to about 175 mcg. A specific patch might be worn for one week and deliver to systemic circulation in that period of time an average daily dose of 70, 100, 125, 140 or 175 mcg of norgestimate or norelgestromin.

In Table 1, there are disclosed preferred oral daily extended cycle contraceptive regimens according to the present invention containing norgestimate (NGM) or norelgestromin (NGMN). A placebo containing no hormone is administered in the off period and a single tablet is administered in the active period containing the hormones as reported.

TABLE 1

| | | Tablet 17α- | Tablet |
|---|---|---|---|
| | Active/Placebo | ethinylestradiol | progestogen |
| Regimen # | days | content | content |
| 1 | 42/7 | 20 mcg | 125 mcg NGM or NGMN |
| 2 | 42/7 | 20 mcg | 180 mcg NGM or NGMN |
| 3 | 42/7 | 20 mcg | 250 mcg NGM or NGMN |
| 4 | 42/7 | 25 mcg | 125 mcg NGM or NGMN |
| 5 | 42/7 | 25 mcg | 180. mcg NGM or NGMN |
| 6 | 42/7 | 25 mcg | 250 mcg NGM or NGMN |
| 7 | 42/7 | 35 mcg | 125 mcg NGM or NGMN |
| 8 | 42/7 | 35 mcg | 180 mcg NGM or NGMN |
| 9 | 42/7 | 35 mcg | 250 mcg NGM or NGMN |
| 10 | 63/7 | 20 mcg | 125 mcg NGM or NGMN |
| 11 | 63/7 | 20 mcg | 180 mcg NGM or NGMN |
| 12 | 63/7 | 20 mcg | 250 mcg NGM or NGMN |
| 13 | 63/7 | 25 mcg | 125 mcg NGM or NGMN |
| 14 | 63/7 | 25 mcg | 180. mcg NGM or NGMN |
| 15 | 63/7 | 25 mcg | 250 mcg NGM or NGMN |
| 16 | 63/7 | 35 mcg | 125 mcg NGM or NGMN |
| 17 | 63/7 | 35 mcg | 180 mcg NGM or NGMN |
| 18 | 63/7 | 35 mcg | 250 mcg NGM or NGMN |
| 19 | 84/7 | 20 mcg | 125 mcg NGM or NGMN |
| 20 | 84/7 | 20 mcg | 180 mcg NGM or NGMN |
| 21 | 84/7 | 20 mcg | 250 mcg NGM or NGMN |
| 22 | 84/7 | 25 mcg | 125 mcg NGM or NGMN |
| 23 | 84/7 | 25 mcg | 180. mcg NGM or NGMN |
| 24 | 84/7 | 25 mcg | 250 mcg NGM or NGMN |
| 25 | 84/7 | 35 mcg | 125 mcg NGM or NGMN |
| 26 | 84/7 | 35 mcg | 180 mcg NGM or NGMN |
| 27 | 84/7 | 35 mcg | 250 mcg NGM or NGMN |
| 28 | 126/7 | 20 mcg | 125 mcg NGM or NGMN |
| 29 | 126/7 | 20 mcg | 180 mcg NGM or NGMN |
| 30 | 126/7 | 20 mcg | 250 mcg NGM or NGMN |
| 31 | 126/7 | 25 mcg | 125 mcg NGM or NGMN |
| 32 | 126/7 | 25 mcg | 180. mcg NGM or NGMN |
| 33 | 126/7 | 25 mcg | 250 mcg NGM or NGMN |
| 34 | 126/7 | 35 mcg | 125 mcg NGM or NGMN |
| 35 | 126/7 | 35 mcg | 180 mcg NGM or NGMN |
| 36 | 126/7 | 35 mcg | 250 mcg NGM or NGMN |

Each of the regimens of Table 1 might be modified by continuing the administration of a NGM or NGMN in a progestogen only tablet for all days of the off period. The dose might be full dose, which is the same as that administered in the active period, or it might be a dose of 125 mcg or it might be a minimized dose of 50 mcg.

In Table 2, there are disclosed preferred contraceptive transdermal regimens or vaginal ring regimens according to the present invention using weekly patches or rings containing norgestimate (NGM) or norelgestromin (NGMN). The weekly patches or rings deliver to systemic circulation the reported average daily dose of NGM or NGMN. No device is administered in the off period.

TABLE 2

| | | Device 17β- | Device |
|---|---|---|---|
| | Device/Off | estradiol delivery | progestogen |
| Regimen # | weeks | rate | delivery rate |
| 37 | 6/1 | 12 mcg | 70 mcg NGM or NGMN |
| 38 | 6/1 | 12 mcg | 100 mcg NGM or NGMN |
| 39 | 6/1 | 12 mcg | 140 mcg NGM or NGMN |
| 40 | 6/1 | 15 mcg | 70 mcg NGM or NGMN |
| 41 | 6/1 | 15 mcg | 100 mcg NGM or NGMN |
| 42 | 6/1 | 15 mcg | 140 mcg NGM or NGMN |
| 43 | 6/1 | 20 mcg | 70 mcg NGM or NGMN |
| 44 | 6/1 | 20 mcg | 100 mcg NGM or NGMN |
| 45 | 6/1 | 20 mcg | 140 mcg NGM or NGMN |
| 46 | 9/1 | 12 mcg | 70 mcg NGM or NGMN |
| 47 | 9/1 | 12 mcg | 100 mcg NGM or NGMN |
| 48 | 9/1 | 12 mcg | 140 mcg NGM or NGMN |
| 49 | 9/1 | 15 mcg | 70 mcg NGM or NGMN |
| 50 | 9/1 | 15 mcg | 100 mcg NGM or NGMN |
| 51 | 9/1 | 15 mcg | 140 mcg NGM or NGMN |
| 52 | 9/1 | 20 mcg | 70 mcg NGM or NGMN |
| 53 | 9/1 | 20 mcg | 100 mcg NGM or NGMN |
| 54 | 9/1 | 20 mcg | 140 mcg NGM or NGMN |
| 55 | 12/1 | 12 mcg | 70 mcg NGM or NGMN |
| 56 | 12/1 | 12 mcg | 100 mcg NGM or NGMN |
| 57 | 12/1 | 12 mcg | 140 mcg NGM or NGMN |
| 58 | 12/1 | 15 mcg | 70 mcg NGM or NGMN |
| 59 | 12/1 | 15 mcg | 100 mcg NGM or NGMN |
| 60 | 12/1 | 15 mcg | 140 mcg NGM or NGMN |
| 61 | 12/1 | 20 mcg | 70 mcg NGM or NGMN |
| 62 | 12/1 | 20 mcg | 100 mcg NGM or NGMN |
| 63 | 12/1 | 20 mcg | 140 mcg NGM or NGMN |
| 64 | 18/1 | 12 mcg | 70 mcg NGM or NGMN |
| 65 | 18/1 | 12 mcg | 100 mcg NGM or NGMN |
| 66 | 18/1 | 12 mcg | 140 mcg NGM or NGMN |
| 67 | 18/1 | 15 mcg | 70 mcg NGM or NGMN |
| 68 | 18/1 | 15 mcg | 100 mcg NGM or NGMN |
| 69 | 18/1 | 15 mcg | 140 mcg NGM or NGMN |
| 70 | 18/1 | 20 mcg | 70 mcg NGM or NGMN |
| 71 | 18/1 | 20 mcg | 100 mcg NGM or NGMN |
| 72 | 18/1 | 20 mcg | 140 mcg NGM or NGMN |

Each of the regimens of Table 2 might be modified by continuing the administration of a NGM or NGMN in a progestogen only device during the off period. The dose might be full dose, which is the same as that administered in the active period, or it might be a dose of 70 mcg or it might be a minimized dose of 30 mcg.

The estrogen and progestogen component are orally administered preferably together in tablets also containing a pharmaceutically acceptable non-toxic carrier, but they can also be administered separately. Suitable carriers include magnesium carbonate, magnesium stearate, talc, lactose, sugar, peptin, dextrin, starch, methylcellulose, sodium carboxylmethylcellulose, and the like. The tablet may also contain one or more substances, which act as diluents, flavoring agents, solubilizers, lubricants, suspending agents, binders, or tablet disintegrating agents as well as encapsulating materials. In general, the active agents are processed, together with the usual additives, vehicles and/or flavor-ameliorating agents normally employed in Galenic pharmacy , in accordance with generally accepted pharmaceutical practices. The hormone containing tablets might also contain nutritional supplements such as, for example, iron supplements, folic acid, calcium, vitamin B₆, vitamin B₁₂, etc. In the manufacture of a typical tablet, the active agents are granulated with spray dried lactose, a lubricating agent and a colorant and compressed.

Oral tablets are preferably packaged in the form of a pharmaceutical kit or package in which the daily dosages are arranged for proper sequential administration. This invention also relates, therefore, to a pharmaceutical unit which contains the tablets of the regimen in a synchronized, fixed sequence, wherein the sequence or arrangement of the dosage units corresponds to the regimen of daily administration.

The estrogen and progestogen component may be transdermally administered, preferably together, by use of a patch. Broadly, patches are devices which contain at a minimum a drug reservoir matrix for holding the drug and metering the drug deposition or delivery to the skin, a backing, and an adhesive layer for adhering the device to the patient. The device may contain other layers such as a drug release rate controlling layer for modulating delivery rate, and the like. The device may contain permeation enhancers to increase the rate of penetration of drugs across the skin. Patches are well known and understood by persons skilled in the art. Patches are now employed in marketed products for the administration of certain progestogens and estrogens. Specific patches and even their application to steroids of the type described herein are described in U.S. Pat. Nos. 5,474,783; 5,656,286; 5,958,446; 6,024,976; 5,252,334; 5,006,342; and 4,906,463.

The estrogen and progestogen component may be intravaginally administered, preferably together, by use of a ring. Broadly, rings are devices having an elastomeric portion or body into which the active steroid is dispersed and which acts as a reservoir and meter for the diffusion of active to the lining of the vagina. The ring may be composed entirely of elastomer with steroid homogenously dispersed throughout as described in U.S. Pat. No. 3,545,397. The ring may have an inert inner core surrounded by an active containing elastomeric layer as described in U.S. Pat. No. 4,012,496. The ring may have an elastomeric active containing inner core surrounded by a thin elastomeric layer initially containing no active. The ring may have an inert core, surrounded by an active containing elastomeric layer and further surrounded by an elastomeric outer layer of variable thickness initially containing no active as described in U.S. Pat. No. 4,292,965. The elastomer, the layered design of the ring, its surface area, the concentration of active, the nature of the active, etc. all combine to determine the release rate of active. Rings are well known and understood by persons skilled in the art. Rings are now employed in marketed products for the administration of certain steroids. Further specific rings and their application to steroids of the type described herein are described in U.S. Pat. Nos. 4,871,543 and 5,188,835.

### Biological Test Methods

Chemicals

[6,7-³H(N)]-estrone sulfate (³H-E₁S), ammonium salt (sp. act. 53 Ci/mmol) and [4-¹⁴C]-estradiol (¹⁴C-E₂) (sp. act. 57 mCi/mmol) were purchased from New England Nuclear Division (DuPont de Nemours, Les Ulls, France). The purity of the radioisotopes was assessed by thin-layer chromatography (TLC) in the appropriate system before use. E₁S, ammonium salt, unlabeled E₁ and E₂, (analytical grade) were obtained from Sigma-Aldrich Chimle, (St Quentin Fallavier, France). 17-deacetylnorgestimate (NGMN; 13-ethyl-17-hydroxy-18,19-dinor-17α-pregn-4-en-20-yn-3-one oxime) was a gift from R. W. Johnson Pharmaceutical Research Institute, Medicinal Chemistry Department, (Raritan, N.J., USA); medroxyprogesterone acetate (MPA, 17α-acetoxy-6α-methylprogesterone) was obtained from Sigma-Aldrich Chimie. All other chemicals were of the highest grade commercially available.

Cell Culture

The hormone-dependent MCF-7 and T-47D human mammary cancer cell lines were grown in Eagle's Minimal Essential Medium (MEM) buffered with 10 mmol/l HEPES (pH 7.6), supplemented with 2 mmol/l L-glutamine, 100 U/ml penicillin-streptomycin and 5% fetal calf serum (FCS) (A.T.G.C., Marne-la-Vallée, France) for T-47D, or 10% FCS for MCF-7 cells, and incubated at 37° C. n a humidified atmosphere of 5% CO₂. Media were changed twice a week. The cells were passed every 10-12 days and replated in 75 cm² flasks (A.T.G.C.) at 3×10⁶ cells/flask. Four days before the experiments, the cells were transferred to MEM containing 5% steroid-depleted treated FCS. The FCS had been treated overnight at 4° C. with dextran-coated charcoal (DCC) (0.1-1% w/v, DCC-FCS). The MCF-7 and T-47D cell lines used herein were deposited in accordance with the Budapest Treaty under the references MCF7_JJPRD and T47D_JJPRD on May 17, 2002 at The Belgian Co-ordinated Collections of Micro-organisms (BCCM), Laboratorium voor Moleculaire Biologie, Universiteit Gent, K. L. Ledeganckstraat 35, B-9000 Gent, Belgium and are publicly available under accession numbers LMBP 5862CB and LMBP 5863CB, respectively.

Isolation and Quantification of [³H]-Estradiol from Human Mammary Cancer Cells Incubated with [³H]-E₁S

Preconfluent cells were incubated for 4 hours at 37° C. in MEM-DCC-FCS with the addition of 5×10⁻⁹ mol/l of [³H]-E₁S, alone (control cells) or in combination with the different compounds: NGMN or MPA, dissolved in ethanol (final concentration<0.2%), at a range of concentrations of 5×10⁻⁵-5×10⁻⁹ mol/l. Control cells received ethanol vehicle only. After 24 hours, the medium was removed, the cells washed twice with ice-cold Hank's Buffered Saline Solution (HBSS, calcium-magnesium-free) (A.T.G.C.) and harvested by scraping. After centrifugation, the pallet was treated with 80% ethanol and the radioactivity extracted for at least 24 h at −20° C. The cellular radioactivity uptake was determined in the ethanolic supernatant and the DNA content in the remaining pellet was evaluated according to Burton Biochem Journal 62:315-323, 1956. [¹⁴C]-E₂ (5,000 dpm) was added to monitor analytical losses and unlabeled E₁ and E₂ (50 μg) were used as carriers and reference indicators. In the total ethanolic extracts, E₂ was isolated by thin layer chromatography (TLC) on silica gel 60F₂₅₄ (Merck, Darmstadt, Germany), developed with chloroform-ethylacetate (4:1, v/v) system. After visualization of the estrogens under U.V. at 254 nm, the appropriate areas were cut off into small pieces, placed in liquid scintillation vials with ethanol (0.5 ml) and allowed to extract for 30 mn. Three ml of Opti-fluor (Packard, Rungis, France) were added and the vials were analyzed for ³H and ¹⁴C contents with quench correction by external standarization. The quantitative evaluation of E₂ was calculated as a percentage of the total radioactivity associated with the cells and then expressed as fmol of E₂ formed /mg DNA from E₁S.

Statistical Analysis

Data are expressed as the mean±standard error of the mean (SEM) values. Student's t-test was used to assess the significance of the differences between means; p values≦0.05 were considered significant.

### Results

Table 3 shows the effects of NGMN and medroxyprogesterone acetate (MPA) concentrations on the conversion of E₁S to E₂ in the hormone-dependent human breast cancer cell line T-47D The data are the mean±SEM of duplicate determinations of 3 independent experiments. *p≦0.05 vs control values (non-treated cells); **p≦0.005 vs control values (non-treated cells)

TABLE 3
T-47D

| | NGMN | MPA |
|---|---|---|
| NGMN or MPA | E₂ formed fmol/mg DNA | E₂ formed fmol/mg DNA |
| conc 1 × 10⁻⁶ mol/l | (% inhibition) | (% inhibition) |
| 0 (control) | 1805 ± 152 (0%) | |
| 0.005 | 1029 ± ? (43 ± 7%)* | 1245 ± ? (31 ± 5%)* |
| 0.5 | 469 ± ? (74 ± 4%)* | 957 ± ? (47 ± 3%)* |
| 50 | 54 ± ? (97 ± 2%)** | 704 ± ? (61 ± 3%)* |

Table 4 shows the effects of NGMN and medroxyprogesterone acetate (MPA) concentrations on the conversion of E₁S to E₂ in the hormone-dependent human breast cancer cell line MCF-7. The data are the mean±SEM of duplicate determinations of 3 independent experiments. *p≦0.05 vs control values (non-treated cells); **p≦0.005 vs control values (non-treated cells)

TABLE 4
MCF-7

| | NGMN | MPA |
|---|---|---|
| NGMN or MPA | E₂ formed fmol/mg DNA | E₂ formed fmol/mg DNA |
| conc 1 × 10⁻⁶ mol/l | (% inhibition) | (% inhibition) |
| 0/control | 2185 ± 101 (0%) | |
| 0.005 | 1639 ± ? (25 ± 4%)* | 2054 ± ? (6 ± 3%)* |
| 0.5 | 940 ± ? (57 ± 5%)* | 1748 ± ? (20 ± 3%)* |
| 50 | 87 ± ? (96 ± 2%)** | 808 ± ? (63 ± 4%)* |

Table 5 shows the IC₅₀ values for NGMN and medroxyprogesterone acetate (MPA) in the conversion of E₁S to E₂ in the hormone-dependent human breast cancer cell lines MCF-7 and T-47D. IC₅₀ values correspond to the 50% inhibition of the conversion of E₁S to E₂ and were determined using non-linear regression analysis.

TABLE 5

IC₅₀,
1 × 10⁻⁶ mol/l

| | T-47D | MCF-7 |
|---|---|---|
| NGMN | 0.0127 | 0.178 |
| MPA | 2.15 | 26.1 |

As noted above, extended administration of contraceptive hormones (also referred to herein as “continuous administration”), whereby there is no drug-free interval after the traditional 21-day cycle of hormone administration, is a common practice among women wishing to delay or prevent withdrawal bleeding. This is often done as a matter of convenience, for example, to prevent withdrawal bleeding during vacation periods or while participating in athletics. In addition to the convenience of delaying withdrawal bleeding, skipping the hormone-free or placebo interval of cyclic administration reduces many menstrual-related symptoms that occur more frequently during the hormone-free interval than during the rest of the cycle. Such symptoms include headaches, pelvic pain, breast tenderness, bloating and swelling.

Extended regimens for administering oral contraceptive hormones have proven to be both well tolerated and effective in preventing pregnancy and in reducing the number of withdrawal bleeding periods experienced over a given course of extended hormone administration. However, as compared to cyclic regimens, continuous oral contraceptive use is associated with an increased incidence of breakthrough bleeding (normal or excessive bleeding requiring sanitary protection) and spotting (light, infrequent bleeding requiring no sanitary protection), especially during the first few months of use. Clinical studies examining the tolerability and acceptance of extended oral contraceptive regimens report that breakthrough bleeding and/or spotting typically occur in approximately one-quarter of the patients evaluated.

It has surprisingly been found that extended transdermal administration of contraceptive hormones results in enhanced compliance, longer median time-to-first bleed, fewer mean bleeding days through day 56, and reduced median incidence of headaches as compared to cyclic transdermal administration. Comparing extended transdermal administration to published data from studies of extended oral administration indicates that transdermal delivery offers superior benefits, not only in bleeding control (absence of vaginal bleeding that requires sanitary protection of at least one pad or tampon per day), but also in continuation and satisfaction rates.. In addition, comparing extended transdermal administration to published data from studies of parenteral hormonal contraceptive delivery systems indicates that the transdermal system offers superior bleeding control to some of these systems, e.g., NORPLANT-2 (Wyeth Pharmaceuticals, Philadelphia, Pa.) and DEPO-PROVERA (Pharmacia, Inc., Peapack, N.J.).

Example I describes a study in which cycle control was compared for extended transdermal administration of contraceptive hormones versus cyclic transdermal administration. The study was conducted using a transdermal contraceptive patch marketed under the trade name ORTHO EVRA and available from Ortho-McNeil Pharmaceuticals, Inc., Raritan, N.J. Each patch provides for the daily administration of 20 μg of ethinyl estradiol and 150 μg of norelgestromin. The patch is fully described in U.S. Pat. Nos. 5,876,746; 5,972,377; and 6,071,531, which are incorporated herein by reference in their entirety.

### EXAMPLE I

A multi-center (10 clinical research sites), open-label study was conducted in which 239 regularly menstruating women were randomized 2:1 to receive, respectively, either norelgestromin/ethinyl estradiol (NGM/EE) extended regimen or NGM/EE cyclic regimen for a 112-day treatment period. There were six clinic visits: one screening visit (up to Day-28), one baseline/randomization visit (Day-7 to -1), and four visits during treatment (Days 28, 56, 84, and 112). Day 1 was the first day (next menses after the baseline visit) on study medication.

Subjects were post-menarcheal, pre-menopausal females 18 to 45 years of age (women between the ages of 35 and 45 were non-smokers), who were menstruating regularly. Eligible participants were required to be in good health as confirmed by medical history and physical/gynecological/breast examinations, Papanicolou smear, and hemoglobin/hematocrit testing performed at screening. Subjects were not pregnant and not lactating. The last term pregnancy had to have been completed at least 42 days prior to the screening visit, and there had to have been at least one normal menstrual period since pregnancy. To be eligible for the study, women agreed not to use any other steroid hormone therapy except topical corticosteroids, if needed, during the trial. In addition, if a subject had undergone uterine surgery or removal of an intrauterine device (IUD), vaginal ring or contraceptive implant or had previously used parenteral depot preparations she was required to have since had at least one normal menstrual period prior to entering the study.

Women were excluded from participation if they had a history or presence of any disorder commonly accepted as a contraindication to steroid hormonal therapy. In addition, because the drug studied had a transdermal application, women with a history of dermal hypersensitivity in response to topical bandage or adhesive applications, or any skin condition affecting the potential sites of patch application were excluded. Treatment with a continuous regimen of an oral contraceptive within the three months prior to screening was exclusionary, as was previous use of any contraceptive patch including NGMN/EE. Persons who were amenorrheic were not allowed entry into the study nor were women who had received DEPO-PROVERA injection or other hormonal injectables in the six months prior to screening; women who currently had NORPLANT or other hormonal implants in place, or had them removed within 60 days prior to screening; or women who had used a steroid-containing IUD within three months prior to screening.

Additional exclusionary conditions included consistently elevated blood pressure; uncontrolled thyroid disorder; a recent history of alcohol or other substance abuse; significant depression or psychiatric disease; or any other medical condition, planned procedure, or concomitant drug use that the investigator thought might put the subject at risk.

Subjects were randomly assigned to receive either the extended or cyclic regimen of NGMN/EE. Subjects assigned to the extended regimen applied one patch weekly for 12 consecutive weeks (84 days) followed by one week (7 days) without patch application (“patch-free”), and then followed by another three consecutive weeks (21 days) of weekly patch applications. In the cyclic treatment regimen there were four consecutive cycles (112 days) in which the subject applied one patch weekly for three weeks (21 days) followed by one week (7 days) patch-free. Subjects were provided with instructions regarding appropriate locations for and method of patch application.

Subjects used a diary throughout the trial to record bleeding and headache information and the site of patch application. Diaries were dispensed at baseline and subjects were instructed to start recording bleeding and headache data from Day 1 (the first day of their next menses after baseline). Information collected from the diaries included whether the patch was worn, if there was spotting or bleeding, how many tampons/pads or pantiliners were used, whether the subject had a headache and its severity, and whether the subject experienced nausea, sensitivity to light, throbbing or one-sided head pain. The diaries were collected by clinic personnel and reviewed with the subject at each visit.

The following definitions were used:

Bleeding=vaginal bleeding that required sanitary protection of at least one pad or tampon per day.

Spotting=vaginal bleeding that did not require sanitary protection (use of pantiliners was acceptable).

Bleeding day=a day on which bleeding was recorded.

Spotting day=a day on which spotting alone was recorded, if spotting and bleeding occurred on the same day, bleeding was the dominant event and the day was recorded as a bleeding day.

Bleeding-free day=a day on which neither bleeding nor spotting was recorded.

Bleeding and/or spotting episode=any set of one or more consecutive bleeding or spotting days bounded by bleeding free days.

Total number of bleeding and/or spotting days=included any bleeding or spotting that occurred between Day 1 and Day 84 (or the last reference period day), minus the number of “rollover” bleeding days from the first menses beginning on Day 1. Total number of breakthrough bleeding and/or spotting days=the number of withdrawal days subtracted from the total number of bleeding and/or spotting days.

The population analyzed, and for which data are shown, are the “perfectly compliant completers” (PCC). This population reflects product performance under ideal circumstances (ie—perfect use). A PCC subject wore the patch every day, as directed, and wore no patch for more than 7 days. In the extended regimen group, this required daily patch wear through 84 days. In the cyclic group, this required daily patch wear for days 1-21, 29-49, and 57-77, and patch-free on days 22-28, 50-56, and 78-84.

Results

The advantages provided by extended transdermal administration of contraceptive hormones versus cyclic transdermal administration, extended oral administration and parenteral delivery systems are evident from the data discussed below.

The data in Table 6 illustrate improved subject compliance with extended transdermal administration as compared to cyclic administration of contraceptive hormones.

TABLE 6
Percent of Subjects with Perfect Compliance by Reference Periods

| | Extended Regimen | | Cyclic Regimen | |
|---|---|---|---|---|
| | (N = 155) | | (N = 80) | |
| | Total | | Total | |
| | Number | | Number | |
| | Completed | Perfect | Completed | Perfect |
| | Interval | Compliance | Interval | Compliance |
| Days 1-28 | 150 | 129 (86.0%) | 77 | 61 (79.2%) |
| Days 29-56 | 137 | 119 (86.9%) | 73 | 57 (78.1%) |
| Days 57-84 | 129 | 112 (86.8%) | 73 | 73 (78.1%) |

Table 7 shows the significantly longer median time-to-first bleed experienced by subjects receiving extended transdermal administration versus cyclic transdermal administration.

TABLE 7
Time to First Bleed
Kaplan-Meier Estimates

| | | | Between |
|---|---|---|---|
| | Extended | Cyclic | Treatment |
| | Administration | Administration | Comparison |
| | (N = 112) | (N = 57 | p-value |
| N | 108 | 57 | <0.0001 |
| Mean | 53.9 | 24.7 | |
| Standard Error | 2.7 | 1.0 | |
| Minimum | 5 | 6 | |
| 25^{th} Percentile | 29.0 | 24.0 | |
| Median | 52.0 | 25.0 | |
| 75^{th} Percentile | 85.0 | 26.0 | |
| Maximum | 108 | 53 | |

Tables 8 and 9 illustrate that subjects receiving extended transdermal administration experienced significantly fewer bleeding-spotting episodes throughout the entire 84 day reference period as compared to subjects receiving cyclic transdermal administration.

TABLE 8
Total Number of Bleeding-Spotting Episodes
for Reference Period Day 1 to 84
(excluding initial bleeding days)

| | Extended | Cyclic | Between |
|---|---|---|---|
| | Regimen | Regimen | Treatment |
| | N-112 | N = 57 | Comparison |
| N | 112 | 57 | |
| Mean | 2.34 | 3.56 | <0.0001 |
| Standard Deviation | 1.83 | 1.67 | |
| Minimum | 0 | 2 | |
| Median | 2.0 | 3.0 | |
| Maximum | 9 | 13 | |

TABLE 9
Summary of the Number of Bleeding-Spotting
Episodes for Selected Reference Periods
(excluding initial bleeding days)

| | | | | Between |
|---|---|---|---|---|
| | | Extended | Cyclic | Treatment |
| | | Regimen | Regimen | Comparison |
| | | (N = 112) | (N = 59) | p-value |
| Day 1 to Day 42 | N | 112 | 59 | |
| | Mean | 0.93 | 1.34 | 0.0072 |
| | Std Dev | 1.00 | 0.80 | |
| | Minimum | 0 | 1 | |
| | Median | 1.0 | 1.0 | |
| | Maximum | 4 | 5 | |
| Day 1 to Day 56 | N | 112 | 59 | |
| | Mean | 1.36 | 2.42 | <0.0001 |
| | Std Dev | 1.27 | 1.19 | |
| | Minimum | 0 | 1 | |
| | Median | 1.0 | 2.0 | |
| | Maximum | 5 | 9 | |
| Day 1 to Day 91 | N | 112 | 59 | |
| | Mean | 2.92 | 3.62 | 0.0140 |
| | Std Dev | 1.83 | 1.64 | |
| | Minimum | 0 | 2 | |
| | Median | 3.0 | 3.0 | |
| | Maximum | 10 | 13 | |
| Day 1 to Day 98 | N | 112 | 59 | |
| | Mean | 3.01 | 3.70 | 0.0223 |
| | Std Dev | 1.83 | 1.93 | |
| | Minimum | 0 | 2 | |
| | Median | 3.0 | 3.0 | |
| | Maximum | 10 | 15 | |
| Day 1 to Day 112 | N | 112 | 59 | |
| | Mean | 3.07 | 4.16 | 0.0006 |
| | Std Dev | 1.87 | 2.02 | |
| | Minimum | 0 | 2 | |
| | Median | 3.0 | 3.9 | |
| | Maximum | 10 | 15 | |

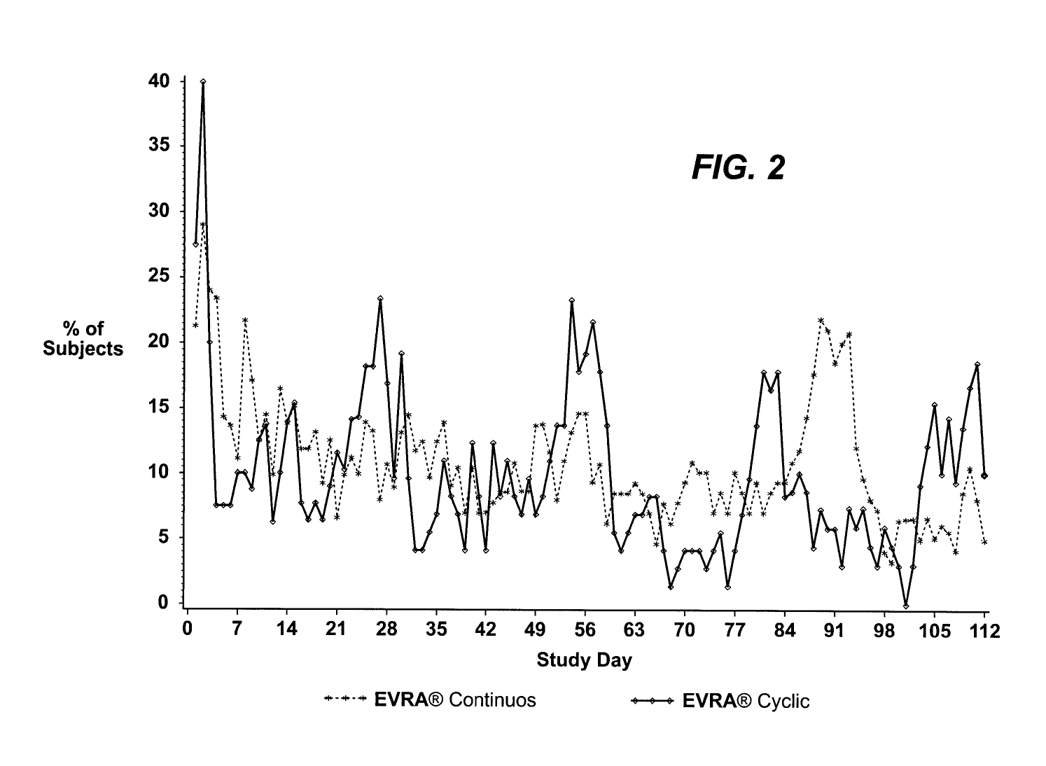

Another benefit of extended hormone dosing is prevention of menstrual-related symptoms, such as headache. The data in Table 10 show that subjects receiving extended transdermal administration had fewer median headache days over time in comparison to the cyclic users. In addition, it appears that headache frequency increases when hormone dosing is withdrawn, as shown in FIG. 2. Thus, there may be other medical benefits to continuous dosing which have not as yet been specifically identified, and it may also be the case that the withdrawal of hormone triggers pathologic symptoms (catamenial seizures, epilepsy, endometriosis, and premenstrual symptoms).

TABLE 10
Headache Data

| | Extended Administration | | | | Cyclic Administration | | | |
|---|---|---|---|---|---|---|---|---|
| Variable | 1-28 | 29-56 | 57-84 | 1-91 | 1-28 | 29-56 | 57-84 | 1-91 |
| Number of Subjects | 155 | 146 | 131 | 155 | 80 | 73 | 74 | 80 |
| % with Headache | 74.84 | 56.85 | 53.44 | 88.39 | 83.75 | 67.12 | 59.46 | 88.75 |
| # of Headache Days | | | | | | | | |
| Mean | 3.94 | 2.91 | 2.31 | 2.66 | 3.69 | 2.84 | 2.24 | 2.37 |
| Std, Dev. | 4.79 | 4.77 | 3.81 | 4.16 | 3.01 | 3.00 | 2.79 | 2.84 |
| Min.-Max. | 0-28 | 0-28 | 0-27 | 0-28 | 0-13 | 0-12 | 0-14 | 0-14 |
| Median | 3.0 | 1.0 | 1.0 | 1.0 | 3.0 | 2.0 | 2.0 | 1.0 |
| # of migraine days | | | | | | | | |
| Mean | 1.10 | 0.79 | 0.56 | 0.74 | 1.11 | 0.97 | 0.65 | 0.75 |
| Std. Dev. | 1.75 | 1.68 | 1.62 | 1.56 | 1.50 | 1.52 | 1.20 | 1.31 |
| Min.-Max. | 0-10 | 0-13 | 0-16 | 0-16 | 0-9 | 0-7 | 0-6 | 0-9 |
| Median | 0.0 | 0.0 | 0.0 | 0.0 | 1.0 | 0.0 | 0.0 | 0.0 |

The following illustrates the advantages of an extended 56-day transdermal regimen over cyclic transdermal administration and extended oral administration. As used herein, and as understood by those skilled in the art, the term “extended 56-day transdermal regimen” means 56 days of continuous hormone administration followed by a hormone-free period, typically 4-8 days of no hormone administration.

With respect to traditional cyclic transdermal administration, the data set forth above in TABLE 7 show that the median time to first bleed is 52 days with extended administration as compared to 25 days with cyclic administration. This means that the majority of extended regimen users will have delayed bleeding until day 52.

The data set forth below in Table 11 illustrate that over a 56-day period of continuous administration, subjects receiving extended transdermal administration experience fewer mean and median bleeding-spotting days as compared to subjects receiving cyclic administration.

TABLE 11
Summary of the Number of Bleeding-Spotting
Days for Selected Reference Periods
(excluding initial bleeding days)

| | | | | Between |
|---|---|---|---|---|
| | | Extended | Cyclic | Treatment |
| | | Regimen | Regimen | Comparison |
| | | (N = 112) | (N = 57) | p-value |
| Day 1 to Day 42 | N | 112 | 57 | |
| | Mean | 5.26 | 7.04 | 0.0765 |
| | Std Dev | 7.04 | 3.70 | |
| | Minimum | 0 | 3 | |
| | Median | 2.0 | 6.0 | |
| | Maximum | 31 | 22 | |
| Day 1 to Day 56 | N | 112 | 57 | |
| | Mean | 9.55 | 11.56 | 0.1729 |
| | Std Dev | 10.46 | 5.03 | |
| | Minimum | 0 | 4 | |
| | Median | 7.0 | 10.0 | |
| | Maximum | 45 | 31 | |
| Day 1 to Day 91 | N | 112 | 57 | |
| | Mean | 23.85 | 19.14 | 0.0608 |
| | Std Dev | 18.07 | 7.35 | |
| | Minimum | 0 | 8 | |
| | Median | 19.0 | 18.0 | |
| | Maximum | 80 | 45 | |
| Day 1 to Day 98 | N | 112 | 57 | |
| | Mean | 25.88 | 19.35 | 0.0109 |
| | Std Dev | 18.31 | 7.74 | |
| | Minimum | 0 | 8 | |
| | Median | 20.5 | 18.0 | |
| | Maximum | 82 | 47 | |
| Day 1 to Day | N | 112 | 57 | |
| 112 | | | | |
| | Mean | 27.47 | 22.22 | 0.0570 |
| | Std Dev | 19.66 | 8.86 | |
| | Minimum | 0 | 9 | |
| | Median | 21.2 | 20.0 | |
| | Maximum | 95 | 55 | |

Table 12 shows that over a 56-day period of continuous administration, subjects receiving extended transdermal administration experience significantly fewer bleeding days per 28-day interval than subjects using a traditional cyclic regimen.

TABLE 12
Total Number of Bleeding Days by 28-Day Intervals
(excluding initial bleeding days)

| | | | | Between |
|---|---|---|---|---|
| | | Extended | Cyclic | Treatment |
| | | Regimen | Regimen | Comparison |
| | | (N = 112) | (N = 57) | p-value |
| Day 1-28 | N | 112 | 57 | <0.0001 |
| | Mean (SD) | 1.02 (2.51) | 4.14 (2.42) | |
| | Minimum | 0 | 0 | |
| | Median | 0.0 | 4.0 | |
| | Maximum | 13 | 12 | |
| Day 29-56 | N | 112 | 57 | 0.0206 |
| | Mean (SD) | 3.98 (6.25) | 6.00 (2.56) | |
| | Minimum | 0 | 1 | |
| | Median | 0.0 | 6.0 | |
| | Maximum | 28 | 19 | |
| Day 57-84 | N | 112 | 57 | 0.6179 |
| | Mean (SD) | 4.94 (7.07) | 5.42 (1.95) | |
| | Minimum | 0 | 2 | |
| | Median | 2.0 | 5.0 | |
| | Maximum | 28 | 12 | |

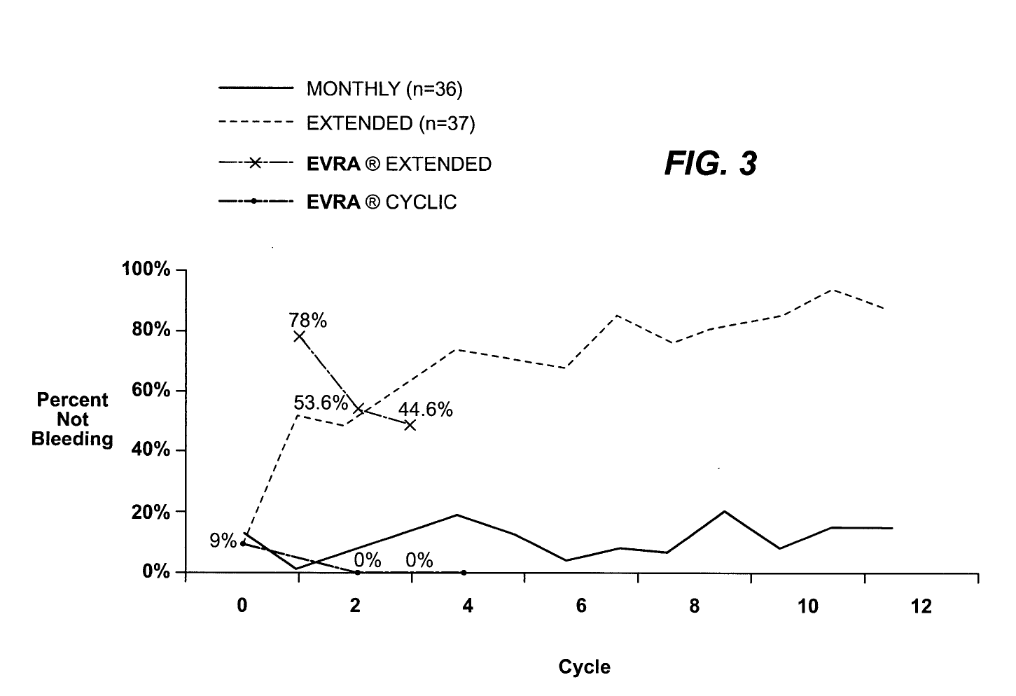

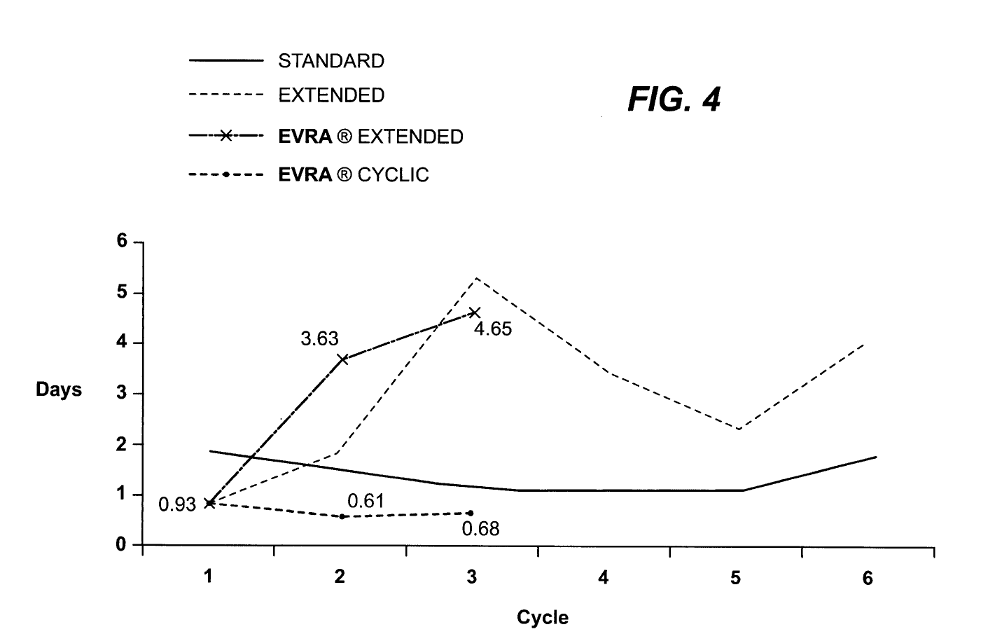

FIGS. 3 and 4 illustrate a comparison between extended transdermal administration and extended oral administration. This comparison is based on data derived from the present study using the ORTHO EVRA transdermal patch and a published study relating to an extended oral regimen using an oral contraceptive marketed under the trade name ALESSE, available from Wyeth Pharmaceuticals, Philadelphia, Pa. Miller, L. et al., Continuous Combination Oral Contraceptive Pills to Eliminate Withdrawal Bleeding: A Randomized Trial, Obstetrics & Gynecology, Vol. 101, No. 4 (April 2003). The ALESSE product is marketed as a 28-day packet. Each packet has 21 tablets, each of which contains 20 μg of ethinyl estradiol and 100 μg of levonorgestrel, and seven hormone-free tablets. The hormone-containing tablets are taken for the first 21 days of each cycle, and the hormone-free tablets are taken on days 22-28 of each cycle In the Miller study, the hormone-free week was eliminated and daily dosing with active hormone was administered for 336 consecutive days.

As shown in FIG. 3, a higher percentage of subjects on the extended transdermal regimen did not require sanitary protection per 28-day interval, through day 56, as compared to the percentage of those not requiring protection in the reported ALESSE study (78% vs 16% for days 1-28; 54% vs 49% for days 29-56).

In another study of ALESSE by Kwiecien et al, ALESSE was administered continuously over 168 days. FIG. 4 illustrates that for extended transdermal administration there were more days per 28-day interval not requiring sanitary protection through day 56, as compared to the number of days not requiring protection reported in the ALESSE study through day 56. Kwiecien et al do report fewer mean number of bleeding days in each treatment cycle that required protection as compared to the results of the present study. However, this appears to result from a small percentage of subjects from the Kwiecien study who reported an abnormally large number of bleeding days.

Extended transdermal administration of contraceptive hormones also presents advantages when compared to extended parenteral delivery systems, such as DEPO-PROVERA (DMPA), NORPLANT-2, and the Levonorgesterol-Intrauterine System, MIRENA, available from Berlex Laboratories, Richmond, Calif. These differ from ORTHO EVRA in that they are all progestogen only-containing devices. Yet, by their design, they are used for extended periods of time. By month three (days 57-84), 25% of ORTH EVRA extended users were amenorrheic. This is in contrast to 10% at month three with DMPA use and 5-10% at month three with NORPLANT-2. Belsey E M, et al. Contraception 1988. 38(2): 181-205; Task Force on Long-Acting Systemic Agents for Fertility Regulation, WHO. Contraception 1987; 35 (6): 591-610); (Qin, et al Contraception 64 (2001), pp 301-303. Published data on bleeding days experienced in the first three months following insertion of the MIRENA device show a mean of approximately 7, 5, and 4 days per 28-day interval through day 84 (Andersson, Odlind, et al. Contraception 1994; 49:56-72). This is in contrast to the approximately 1, 4, and 5 bleeding days seen with extended transdermal use over the same time period (see table 12). While the bleeding days steadily decrease with each successive month of MIRENA use, the first 56 days clearly exhibit more bleeding than seen in the first 56 days of extended transdermal use.

Having described the invention in specific detail and exemplified the manner in which it may be carried into practice, it will be apparent to those skilled in the art that innumerable variations, applications, modifications, and extensions of the basic principles involved may be made without departing from its spirit or scope. It is to be understood that the foregoing is merely exemplary and the present invention is not to be limited to the specific form or arrangements of parts herein described and shown.

What is claimed is:

1. A method of contraception comprising the step of:
administering to a menstruating female a cycle of contraceptive therapy, each cycle of therapy including the transdermal administration of a combination of ethinyl estradiol and norelgestromin for at least 42 successive days, wherein said method provides enhanced bleeding control and enhanced continuation and satisfaction rates in menstruating females using said method.

2. The method of claim 1, wherein each cycle of therapy includes 56 consecutive days of therapy.

3. The method of claim 2, wherein each cycle includes a hormone-free period following the 56 days of continuous therapy.

4. The method of claim 3, wherein the hormone-free period is from 4-8 days.

5. The method of claim 1, wherein 20 μg of ethinyl estradiol and 150 μg of norelgestromin are administered daily.

6. A contraceptive therapy unit adapted for administration in an extended contraceptive regimen, said unit comprising a sufficient number of transdermal patches to provide at least 42 consecutive days of contraceptive therapy, wherein each transdermal patch comprises a suitable matrix and a combination of ethinyl estradiol and norelgestromin, and wherein said regimen provides enhanced bleeding control and enhanced continuation and satisfaction rates in menstruating females using said regimen.

7. The unit of claim 6, wherein said unit includes 8 transdermal patches

8. The unit of claim 4, wherein each patch provides for the daily administration of 20 μg of ethinyl estradiol and 150 μg of norelgestromin.
